(19) United States
(12) Patent Application Publication
(10) Pub. No.: US 2023/0404554 A1
(43) Pub. Date: Dec. 21, 2023

(54) VAGINAL SCOPE

(71) Applicant: REGENTS OF THE UNIVERSITY OF MICHIGAM, Ann Arbor, MI (US)

(72) Inventors: Sandra Dubaisi, Findlay, OH (US); Julia Greenspan, New City, NY (US); Jessica Jonah, Boston, MA (US); Sara Staley, Stamford, CT (US); Allison Wilcox, Minneapolis, MN (US); Marilyn S. Filter, Saginaw, MI (US); Lyn Behnke, Tawas City, MI (US); Line van Niewstadt, Ann Arbor, MI (US)

(21) Appl. No.: 18/210,954

(22) Filed: Jun. 16, 2023

Related U.S. Application Data
(60) Provisional application No. 63/353,288, filed on Jun. 17, 2022.

(51) Int. Cl. A61B 10/04; A61B 1/303; A61B 1/31; A61B 1/00

(52) U.S. Cl. CPC A61B 10/04; A61B 1/303; A61B 1/31; A61B 1/00066; A61B 1/00097; A61B 1/00154; A61B 2010/0216

(57) ABSTRACT

The present disclosure relates to an apparatus for conducting gynecological or proctological exams. The apparatus including a sensor body having a longitudinal axis, a proximal end, a distal end, a first passage disposed coaxial with the longitudinal axis and passing through the sensor body from the proximal end to the distal end, and a second passage including a first opening disposed proximate the distal end of the sensor body. Additionally, the apparatus includes a grip disposed on the proximal end of the sensor body, the grip including a second opening of the second passage. Further, the present disclosure provides a brush control mechanism having a proximal end and a distal end, the proximal end of the brush control mechanism configured to pass through the first passage from the distal end of the sensor body; and a brush disposed on the distal end of the brush control mechanism.

### CROSS-REFERENCE TO RELATED APPLICATIONS

The present application claims the benefit of the filing date of U.S. Provisional Patent Application No. 63/353,288, filed Jun. 17, 2022. The contents of U.S. Provisional Application No. 63/353,288 is hereby incorporated by reference in its entirety.

### FIELD

This application relates generally to apparatuses and techniques for conducting perineal exams, and more particularly, to an apparatus for conducting gynecological and proctological exams and methods of using the same.

### BACKGROUND

Numerous medical examinations require the examination and tissue collection from inside a patient's body. For example, gynecological and proctological examinations include evaluation and tissue collection for a patient's rectum, vagina, and/or cervix. For example, typical gynecological examinations require a patient to sit with their legs elevated and spread in stirrups. A speculum is then used to mechanically stretch the vagina open and permit a medical practitioner to examine and collect tissue samples from the vagina and/or rectum, though require the practitioner to be in close proximity to the patient's genitalia.

The invasiveness and discomfort of these examinations cause many people to avoid these examinations. Avoiding such examinations can permit conditions, such as cancers or sexually transmitted diseases, to go undiagnosed. Leaving such conditions undiagnosed can be very detrimental to the health and life of a patient. Conducting routine examinations is the best method to determine early disease diagnosis or abnormalities. Accordingly, to improve patient compliance, there is a need for improved devices and methods that facilitate an atraumatic examination experience.

### SUMMARY

In one aspect, the present disclosure provides an apparatus for conducting gynecological or proctological exams. The apparatus including a sensor body having a longitudinal axis, a proximal end, a distal end, a first passage disposed parallel with the longitudinal axis and passing through the sensor body from the proximal end to the distal end, and a second passage including a first opening disposed proximate the distal end of the sensor body. Additionally, the apparatus includes a grip disposed on the proximal end of the sensor body, the grip including a second opening of the second passage. Further, the present disclosure provides a brush control mechanism having a proximal end and a distal end, the proximal end of the brush control mechanism configured to pass through the first passage from the distal end of the sensor body; and a brush disposed on the distal end of the brush control mechanism.

In some variations, the second passage is configured to receive an endoscope. Additionally, the endoscope may further include a light source and a camera.

In other variations, the sensor body can include an alignment guide and the brush control mechanism can further include an alignment protrusion. The alignment guide of the sensor body may be configured to receive the alignment protrusion of the brush control mechanism and rotate the brush control mechanism. Also, the alignment guide can further includes a stop configured to inhibit movement of the brush control mechanism. For example, the stop may be configured to inhibit movement of the brush control mechanism from further progressing into the patient's body.

In further alternative arrangements, the sensor body can include a lip extending from the distal end of the sensor body. Also, the first passage may define a first length and the brush control mechanism can include a rod having a second length, longer than the first length. Further, the grip can include a first handle and a second handle. In such examples, at least one of the first handle and the second handle is disposed perpendicular to the longitudinal axis. Additionally, the brush may include a tissue swab.

In yet another aspect, the present disclosure provides another apparatus for conducting gynecological and proctological exams. Additionally, the apparatus includes a sensor body having a proximal end, a distal end, a first passage passing through the sensor body from the proximal end to the distal end, and a lip disposed on the distal end of the sensor body. Additionally, a brush control mechanism of the apparatus includes a proximal end and a distal end, the proximal end of the brush control mechanism configured to pass through the first passage from the distal end of the sensor body. Further, the disclosure provides a brush disposed on the distal end of the brush control mechanism.

In some variations, the apparatus includes a grip disposed on the proximal end of the sensor body. In such examples, the grip includes a first handle and a second handle. Further, at least one of the first handle and the second handle can be disposed perpendicular to a longitudinal axis.

In other embodiments, a second passage including a first opening disposed proximate the distal end of the sensor body and a second opening disposed on the grip. The example second passage can be configured to receive an endoscope and the endoscope may comprising a light source and a camera.

In additional variations, the sensor body can comprise an alignment guide and the brush control mechanism can include an alignment protrusion. In such variations, the alignment guide of the sensor body is configured to receive the alignment protrusion of the brush control mechanism and rotate the brush control mechanism. Furthermore, the alignment guide can include a stop configured to inhibit movement of the brush control mechanism.

In yet further variations, the first passage of the apparatus defines a first length and the brush control mechanism defines a rod having a second length. The example second length can be, in some examples, longer than the first length. Also, the brush of the apparatus can further comprise a tissue swab.

In yet another aspect, the present disclosure provides another apparatus for conducting gynecological or proctological exams. The disclosure provides an apparatus including a sensor body having a longitudinal axis, a proximal end, a distal end, a first passage disposed coaxial with the longitudinal axis and passing through the sensor body from the proximal end to the distal end. The apparatus further includes a second passage including a first opening disposed proximate the distal end of the sensor body. The sensor body may further include a lip disposed on the distal end of the sensor body and a grip disposed on the proximal end of the sensor body. The example grip on the sensor body may include a second opening of the second passage. The apparatus may further include a brush control mechanism having a proximal end and a distal end, the proximal end of the brush control mechanism configured to pass through the first passage from the distal end of the sensor body, and a brush disposed on the distal end of the brush control mechanism.

### BRIEF DESCRIPTION OF THE DRAWINGS

The features of this disclosure which are believed to be novel are set forth with particularity in the appended claims. The present disclosure may be best understood by reference to the following description taken in conjunction with the accompanying drawings, in which like reference numerals identify like elements in the several figures, in which:

FIG. 1 is a perspective view of a vaginal scope constructed in accordance with the teachings of the present disclosure.

FIG. 2 is a perspective view of the example vaginal scope of FIG. 1 including a partial cutaway.

FIG. 3 is a side cross sectional view of the example vaginal scope of FIG. 1.

FIG. 4 is a top cross sectional view of the example vaginal scope of FIG. 1.

FIG. 5 is a front cross sectional view of the example vaginal scope of FIG. 1.

FIG. 6 is an exploded view of a second vaginal scope constructed in accordance with the teachings of the present disclosure.

FIG. 7 is a side cross sectional view of the example vaginal scope of FIG. 6.

FIG. 8 is a front cross sectional view of the example vaginal scope of FIG. 6.

FIG. 9 is a side view of an example brush useable in the example vaginal scope of FIGS. 1 and 6.

The figures depict preferred embodiments for purposes of illustration only and are not to scale. One skilled in the art will readily recognize from the following discussion that alternative embodiments of the systems and methods illustrated herein may be employed without departing from the principles of the invention described herein.

### DETAILED DESCRIPTION

Current medical examination techniques for gynecological, rectal, and proctological examinations require patients to, typically, assume an uncomfortable examination position to provide the medical practitioner sufficient space to observe and operate the medical tools necessary for the examination. For example, gynecological exams often require female patients to take an uncomfortable and vulnerable position on their back with their legs up and spread in stirrups. Such positions can be particularly difficult for survivors of sexual assault.

Additionally, not all medical practitioners follow universal precautions in gynecological, rectal, and/or proctological examinations. Certain examinations require medical practitioners to place their face near the examination area. As a result, during conventional examinations, the medical practitioner may get human discharge on their clothing, gloves, and/or face. Such contamination risks the health of the medical practitioner and other patients as well.

The scope of the present disclosure improves the examination procedure for both the patient and the medical practitioner. First, the vaginal scope allows the patient to undergo the examination from either a lateral or dorsal position without the use of stirrups. Additionally, the vaginal scope minimizes the need to uncomfortably and mechanically stretch any patient body parts, such as the vagina with the use of a speculum. Further, the scope improves the medical practitioner's examination by providing safer and more informational examinations and procedures. Images collected using the apparatus of the present disclosure may also be stored and transmitted for telehealth consultation or added to a patient's electronic medical records.

From the foregoing benefits, the scope can revolutionize numerous medical fields. For example, the vaginal scope can improve women's healthcare. Because many women avoid gynecological examinations due to discomfort and pain, the improved patient experience provided by the vaginal scope can decrease the number of women who are not up to date on their critical examinations. Furthermore, the vaginal scope can be used for preterm labor to avoid stimulation of the cervix or during emergency department visits involving vaginal complaints. Lastly, the vaginal scope provides a better examination procedure that would more amenable to women who have been sexually assaulted to avoid feelings a further violation.

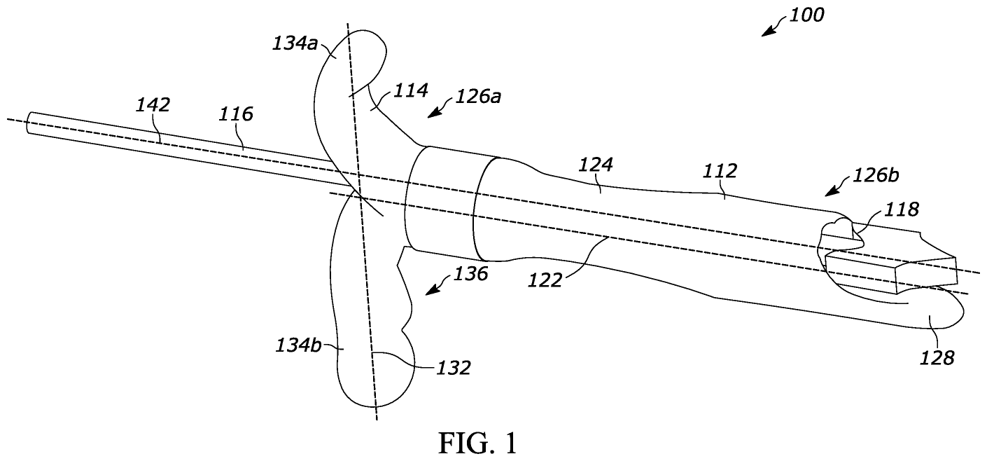

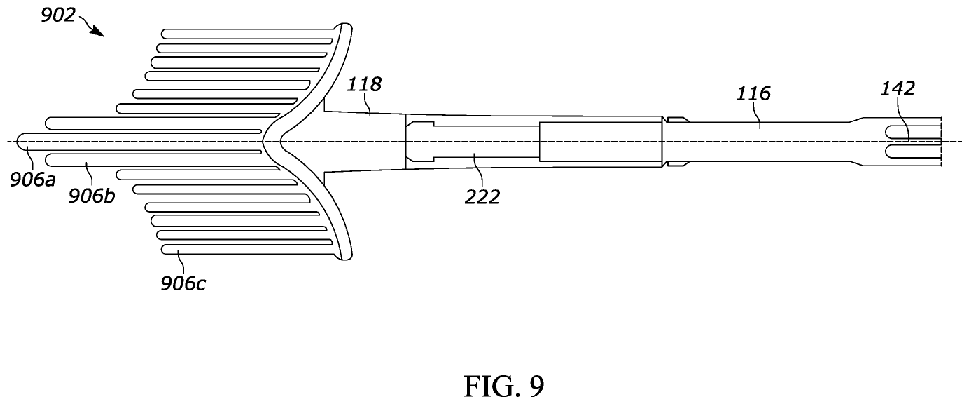

FIG. 1 illustrates a vaginal scope 100 constructed in accordance with the teachings of the present disclosure. As illustrated, the vaginal scope includes a sensor body 112, a grip 114, a brush control mechanism 116, and a brush 118. As shown, the sensor body 112 and the grip 114 are integrally formed together. In some examples, the sensor body 112 and the grip 114 are separate components and coupled together. Additionally, the brush 118 may comprise a tissue swab (discussed in greater detail in connection with FIG. 9) to collect tissue cells for subsequent laboratory analysis.

As shown, the sensor body 112 is disposed about a longitudinal axis 122 and generally defines an approximately cylindrical body 124 disposed between a proximal end 126a and a distal end 126b. As shown, the cylindrical body 124 includes a smooth surface and gentle curves, however, in some examples, the cylindrical body 124 could be a straight cylinder or include different curves. Additionally, the cylindrical body 124 is made from a synthetic polymer, however, the cylindrical body could be made of any medical grade material including natural materials, metals, glass, and mixed polymers. Additionally, disposed on a distal end 126b of the sensor body 112, includes a lip 128 to facilitate the vaginal scope 100 into a patient's vagina or rectum.

The grip 114 of the vaginal scope 100 is disposed on the proximal end 126a of the sensor body 112. The grip 114 can be disposed along a lateral axis 132, disposed perpendicular to the longitudinal axis 122. In other examples, the lateral axis 132 may be disposed at any angle relative the longitudinal axis 122. As shown, the grip 114 includes a first handle 134a and a second handle 134b. As shown, the first handle 134a and the second handle 134b are disposed along the lateral axis 132 and perpendicular to the longitudinal axis 122. Additionally, the grip 114 further includes finger grooves 136 on the second handle 134b, to increase comfort in handling the vaginal scope 100. In other examples, the first handle 134a and the second handle 134b could include more or fewer features for comfort in handling the vaginal scope 100.

The vaginal scope 100 further includes the brush control mechanism 116 and the brush 118. As shown, the brush control mechanism 116 and the brush 118 are disposed along a longitudinal axis 142. As shown, the longitudinal axis 122 and the longitudinal axis 142 are parallel. However, in some examples, the longitudinal axis 122 may be disposed at an angle relative to longitudinal axis 142. Further, in some examples, the longitudinal axis 122 may be coaxial with the longitudinal axis 142.

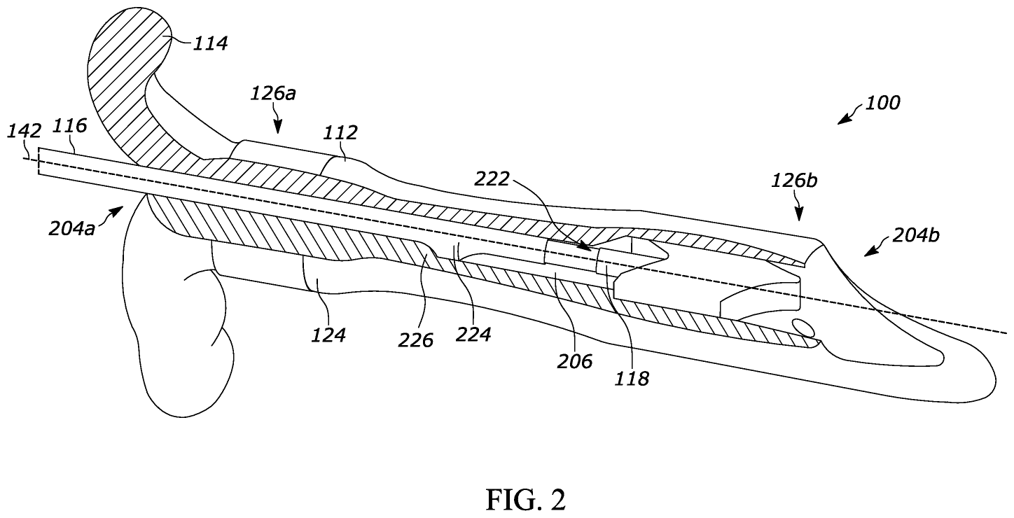

FIG. 2 illustrates the example vaginal scope 100 of FIG. 1, including a partial cutaway. As shown, sensor body 112 includes a proximal opening 204a, a distal opening 204b, and a first passage 206 disposed between the proximal and distal openings 204a, 204b. In the present example, the brush control mechanism 116 is inserted into the sensor body 112 via the distal opening 204b. In other examples, the brush 118 and the brush control mechanism 116 can be inserted into the sensor body 112 through the proximal opening 204a. The first passage 206 defines a first length, the brush control mechanism 116, defining a rod having a second length. In various examples, the second length of the rod is longer than the first length of the first passage.

As illustrated, the brush control mechanism 116 includes a connector 222 to couple the brush control mechanism 116 to the brush 118. However, in some examples, the brush control mechanism 116 and the brush 118 may be integrally formed. The brush control mechanism 116 further includes an alignment protrusion 224 configured to engage an alignment guide 226 disposed in the cylindrical body 124 of the sensor body 112. The example alignment guide 226 may include a stop configured to inhibit longitudinal movement of the brush control mechanism 116. As a result, when the alignment protrusion 224 fully engages the alignment guide 226, the brush control mechanism 116 and the brush 118 are disposed in a storage state within the sensor body 112, the storage state illustrated in FIG. 2. The brush 118 is fully disposed within the sensor body 112 in the storage state. The alignment guide 226 is discussed in greater detail in connection with FIG. 5.

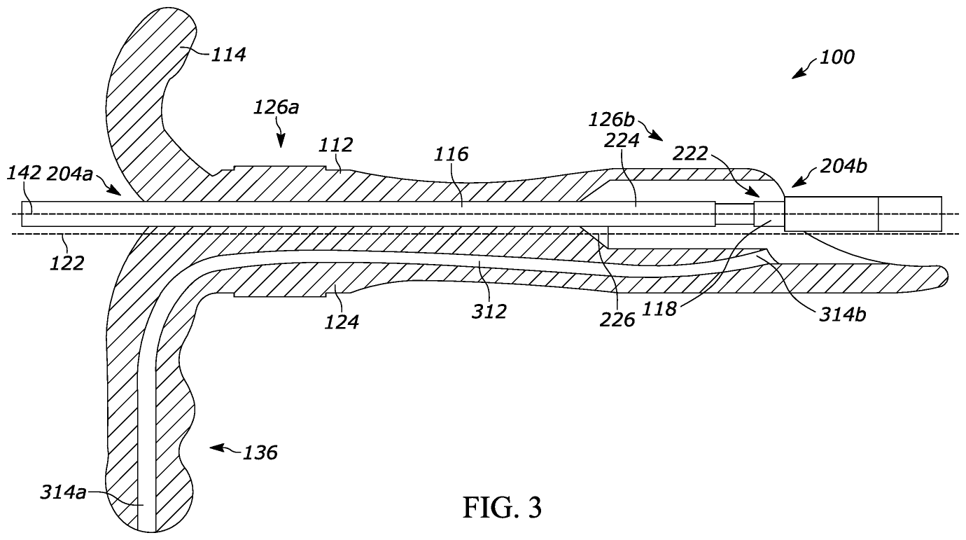

FIG. 3 illustrates the example vaginal scope 100 of FIG. 1, including a side cross sectional view. The example vaginal scope 100 further includes a second passageway 312 disposed between a first opening 314a and a second opening 314b. The second passageway 312 can receive a camera (e.g., endoscope) and/or light source. In such an example, the camera and/or light source can be disposed proximate the second opening 314b to assist a doctor or nurse in controlling the vaginal scope 100. For example, the camera can provide live video to a monitor such that the medical professional can precisely control the vaginal scope 100 and brush 118.

As shown in FIG. 3, the brush 118 extends outside the sensor body 112 in a deployed state. In this position, the brush 118 can interact with the patient's body to collect a tissue sample with bristles (discussed in greater detail in connection with FIG. 9). The brush 118 can be configured to collect samples from a patient's vagina, cervix, colon, or other body part. The brush 118 and the tissue swab may be made of any medical grade material including natural materials, synthetic polymers, metals, and mixtures of polymers.

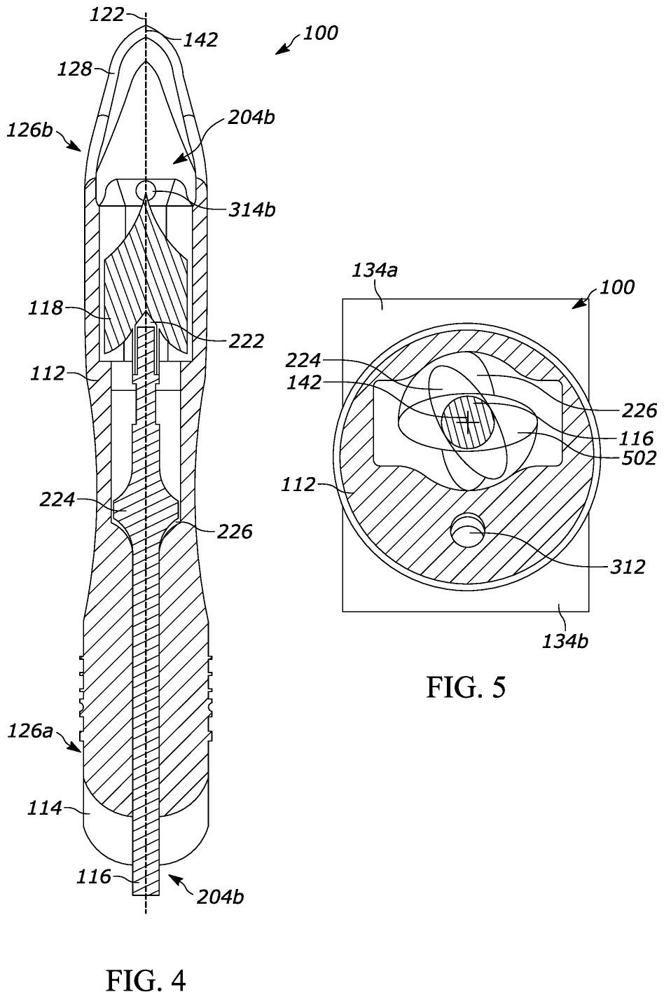

FIG. 4 illustrates the example vaginal scope 100 of FIG. 1, including a top cross sectional view. As shown in FIG. 4, the brush 118 and the alignment protrusion 224 are symmetrical about the longitudinal axis 142, however in various other examples, the brush 118 and the alignment protrusion could be symmetrical about another axis or asymmetrical about any axis.

As shown in the cross section in FIG. 4, the brush control mechanism 116 is partially inserted into the brush 118 at the connector 222. The connector 222 could utilize any known fastening method. For example, the brush control mechanism 116 could be press fit or interference fit into the brush 118. Alternatively, the brush control mechanism 116 could screw into the brush 118. Further, the brush control mechanism 116 and the brush 118 could be secured together via a permanent or temporary adhesive. For example, the temporary adhesive could provide partial resistance to separating the brush 118 from the brush control mechanism 116.

FIG. 5 illustrates the example vaginal scope 100 of FIG. 1, including a front cross sectional view. As shown, the alignment guide 226 includes an angular twist about the longitudinal axis 142. In various examples, the alignment guide 226 may include a sharper or a gentler angular twist. In the present disclosure, the alignment guide 226 is provided to properly orient the brush 118 in the sensor body 112 and to facilitate insertion of the brush control mechanism 116 and the brush 118 into the sensor body 112. The alignment guide 226 ends at a stop 502 to inhibit movement of the brush control mechanism.

In accordance with the present disclosure, the vaginal scope 100 is configured to facilitate examination of internal body parts, such as a vagina, cervix, or colon. Prior to the insertion of the vaginal scope 100, a medical professional (e.g., a doctor, nurse) can insert the brush control mechanism 116 and the brush 118 into the vaginal scope 100 through the distal opening 206b. In use, the vaginal scope 100 is gently inserted into the body of a patient. For example, the lip 128 can facilitate comfortable insertion of the vaginal scope 100 into the body of the patient. With the vaginal scope 100 inserted into the patient, the medical professional can push the brush 118 from the storage state within the sensor body 112 to a deployed state outside the sensor body 112. As a result, the brush 118, including a tissue swab (discussed in greater detail in connection with FIG. 9), can collect a tissue sample from the vagina, cervix, or colon. After collecting the tissue sample, the medical professional can retract the brush 118 back into the storage state to safely retract the brush 118 and tissue swab for subsequent laboratory analysis.

To improve control of the vaginal scope 100 within the patient body, the medical professional may insert an endoscope and light source into the second passageway 312. The endoscope and light source can be configured to provide a live stream video of the patients vagina or rectum. This also assists the medical professional to accurately collect a tissue sample with the brush 118. Additionally, the second opening 314b is disposed at an angle to best view movement of the brush 118 relative to the sensor body 112.

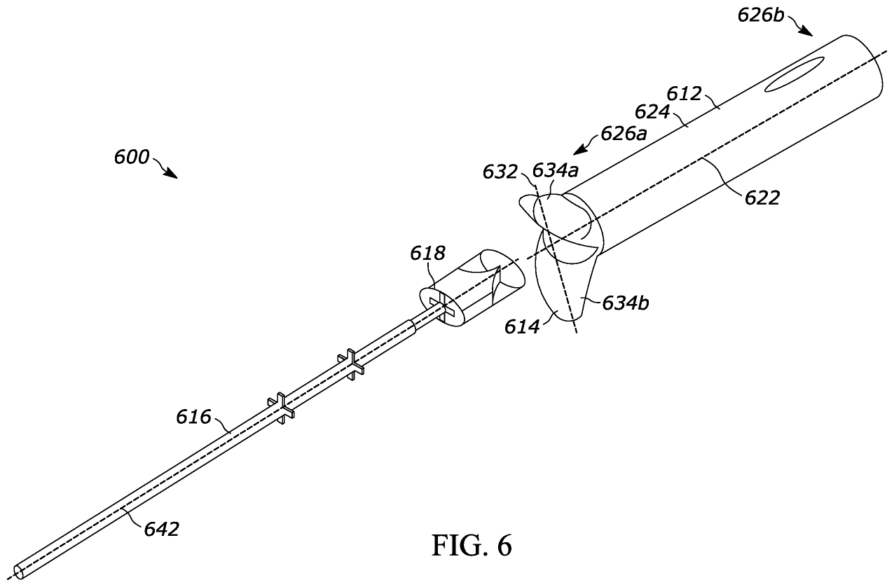

FIG. 6 illustrates an exploded view of a second vaginal scope 600 in accordance with the teachings of the present disclosure. As illustrated, the vaginal scope 600 includes a sensor body 612, a grip 614, a brush control mechanism 616, and a brush 618. As shown, the sensor body 612 and the grip 614 are integrally formed together. In some examples, the sensor body 612 and the grip 614 are separate components and coupled together.

As shown, the sensor body 612 is disposed about a longitudinal axis 622 and generally defines an approximately cylindrical body 624 disposed between a proximal end 626a and a distal end 626b. As shown, the cylindrical body 624 includes a smooth, straight surface, however, in some examples, the cylindrical body 624 could be include gentle curves. Additionally, the cylindrical body 624 is made from a synthetic polymer, however, the cylindrical body could be made of any medical grade material including natural materials, metals, glass, and mixed polymers. Additionally, in some examples, the sensor body 612 includes a lip to facilitate insertion of the vaginal scope 600 into a patient's vagina or rectum.

The grip 614 of the vaginal scope 600 is disposed on the proximal end 626a of the sensor body 612. The grip 614 can be disposed along a lateral axis 632, disposed perpendicular to the longitudinal axis 622. In other examples, the lateral axis 632 may be disposed at any angle relative the longitudinal axis 622. As shown, the grip 614 includes a first handle 634a and a second handle 634b. As shown, the first handle 634a and the second handle 634b are disposed along the lateral axis 632 and perpendicular to the longitudinal axis 622. In some examples, the first handle 634a and the second handle 634b could include features for comfort in handling the vaginal scope 600.

The vaginal scope 600 further includes the brush control mechanism 616 and the brush 618. As shown, the brush control mechanism 616 and the brush 618 are disposed along a longitudinal axis 642. As shown, the longitudinal axis 622 and the longitudinal axis 642 are parallel. However, in some examples, the longitudinal axis 622 may be disposed at an angle relative to longitudinal axis 642. Further, in some examples, the longitudinal axis 622 may be coaxial with the longitudinal axis 642.

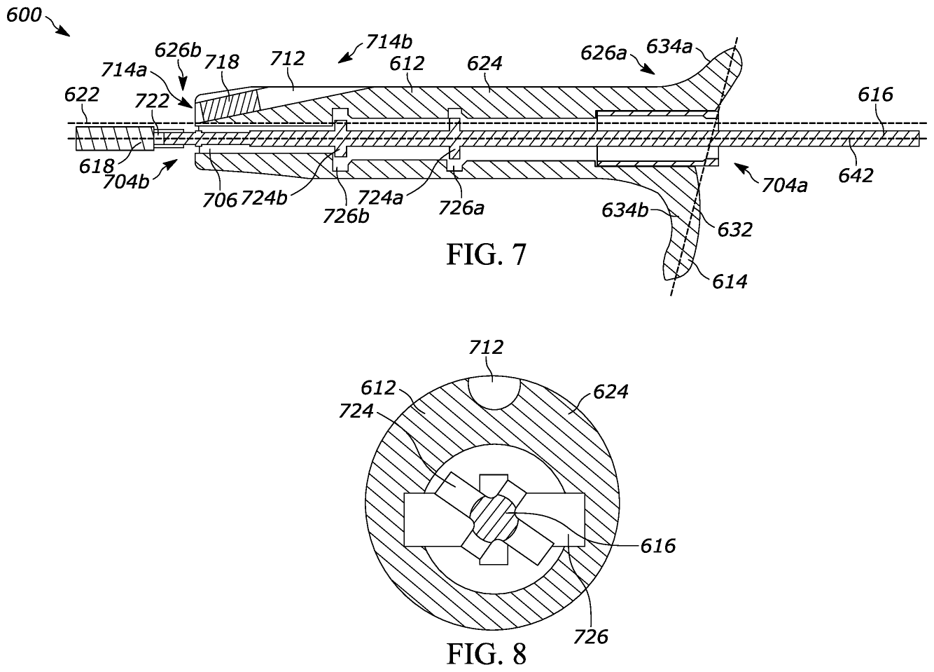

FIG. 7 illustrates the example vaginal scope 600 of FIG. 6, including a side cross sectional view. As shown, sensor body 612 includes a proximal opening 704a, a distal opening 704b, and a bore 706 disposed between the proximal and distal openings 704a, 704b. In the present example, the brush control mechanism 616 is inserted into the sensor body 612 via the proximal opening 704a. In other examples, the brush 618 and the brush control mechanism 616 can be inserted into the sensor body 612 through the distal opening 704b.

As illustrated, the brush control mechanism 616 includes a connector 722 to couple the brush control mechanism 616 to the brush 618. However, in some examples, the brush control mechanism 616 and the brush 618 may be integrally formed. The brush control mechanism 616 further includes alignment protrusions 724a, 724b configured to engage alignment guides 726a, 726b disposed in the cylindrical body 624 of the sensor body 612. The example alignment guides 726a, 726b are configured to control longitudinal movement of the brush control mechanism 616. As a result, when the alignment protrusions 724a, 724b fully engage the alignment guides 726a, 726b, the brush control mechanism 616 and the brush 618 are locked in either a storage state or a deployed state. Alternatively, when the alignment protrusions 724a, 724b are disengaged from the alignment guides 726a, 726b, the brush control mechanism 616 can be moved through the sensor body 612. As shown in FIG. 7, the brush 618 is disposed outside the sensor body 612 in the deployed state. The alignment guides 726 are discussed in greater detail in connection with FIG. 8.

FIG. 8 illustrates the example vaginal scope 600 of FIG. 6, including a front cross sectional view. As shown, the alignment guides 726a, 726b include apertures identically shaped to the alignment protrusions 726a, 726b. In various examples, the alignment guide 726 may include an angular twist. In the present disclosure, the alignment guide 726 is to facilitate controlled insertion of the brush control mechanism 616 and the brush 618 out of the sensor body 612. For example, in some examples the alignment guide 726 and the alignment protrusion 724 are configured to inhibit the brush control mechanism from progressing too far into the patient's body.

FIG. 9 illustrates an example tissue swab useable in connection with the vaginal scope 100, 600 in accordance with the teachings of the present disclosure. For example, the brush 118 could be similar to a single-use cervical cytology brush. As illustrated, the brush 118 includes a plurality of bristles 902 having a subset of first sized bristles 906a, a subset of second sized bristles 906b, and a subset of third sized bristles 906c. In various examples, the size of the subset of bristles 906a, 906b, 906c may vary in length and/or thickness. Further, in other examples, the plurality of bristles 902 may be of uniform size. In various examples, the bristles 902 can be made of any material compatible with collecting a tissue sample, including natural materials (e.g., natural fibers) and synthetic materials (e.g., polymers, silicone, fiberglass).

The arrangement of bristles 902 illustrated in FIG. 9 is provided by way of example. The brush 118 of the present disclosure could have any shape or configuration compatible with collecting a tissue sample. For example, any known biopsy or cytology brush could be adapted for use in the vaginal scope 100, 600 made in accordance with the teachings of the present disclosure.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

What is claimed is:

1. An apparatus for conducting gynecological or proctological exams, comprising:
a sensor body having a longitudinal axis, a proximal end, a distal end, a first passage disposed parallel with the longitudinal axis and passing through the sensor body from the proximal end to the distal end, and a second passage including a first opening disposed proximate the distal end of the sensor body;
a grip disposed on the proximal end of the sensor body, the grip including a second opening of the second passage;
a brush control mechanism having a proximal end and a distal end, the proximal end of the brush control mechanism configured to pass through the first passage from the distal end of the sensor body; and
a brush disposed on the distal end of the brush control mechanism.

2. The apparatus of claim 1, wherein the second passage is configured to receive an endoscope.

3. The apparatus of claim 2, the endoscope further comprising a light source and a camera.

4. The apparatus of claim 1, wherein the sensor body further comprises an alignment guide and the brush control mechanism further comprises an alignment protrusion,
wherein the alignment guide of the sensor body is configured to receive the alignment protrusion of the brush control mechanism and rotate the brush control mechanism.

5. The apparatus of claim 4, wherein the alignment guide further includes a stop configured to inhibit movement of the brush control mechanism.

6. The apparatus of claim 5, wherein the stop is configured to inhibit movement of the brush control mechanism from further progressing into the patient's body.

7. The apparatus of claim 5, the sensor body further comprising a lip extending from the distal end of the sensor body.

8. The apparatus of claim 1, wherein the first passage defines a first length and the brush control mechanism defines a rod having a second length, longer than the first length.

9. The apparatus of claim 1, wherein the grip includes a first handle and a second handle.

10. The apparatus of claim 9, wherein at least one of the first handle and the second handle is disposed perpendicular to the longitudinal axis.

11. The apparatus of claim 1, wherein the brush comprises a tissue swab.

12. An apparatus for conducting gynecological and proctological exams, comprising:
a sensor body having a proximal end, a distal end, a first passage passing through the sensor body from the proximal end to the distal end, and a lip disposed on the distal end of the sensor body;
a brush control mechanism having a proximal end and a distal end, the proximal end of the brush control mechanism configured to pass through the first passage from the distal end of the sensor body; and
a brush disposed on the distal end of the brush control mechanism.

13. The apparatus of claim 12, further comprising a grip disposed on the proximal end of the sensor body.

14. The apparatus of claim 13, wherein the grip includes a first handle and a second handle.

15. The apparatus of claim 14, wherein at least one of the first handle and the second handle is disposed perpendicular to a longitudinal axis.

16. The apparatus of claim 13, further comprising a second passage including a first opening disposed proximate the distal end of the sensor body and a second opening disposed on the grip.

17. The apparatus of claim 16, wherein the second passage is configured to receive an endoscope.

18. The apparatus of claim 17, the endoscope further comprising a light source and a camera.

19. The apparatus of claim 12, wherein the sensor body further comprises an alignment guide and the brush control mechanism further comprises an alignment protrusion,
wherein the alignment guide of the sensor body is configured to receive the alignment protrusion of the brush control mechanism and rotate the brush control mechanism.

20. The apparatus of claim 19, wherein the alignment guide further includes a stop configured to inhibit movement of the brush control mechanism.

21. The apparatus of claim 12, wherein the first passage defines a first length and the brush control mechanism defines a rod having a second length, longer than the first length.

22. The apparatus of claim 12, wherein the brush comprises a tissue swab.

23. An apparatus for conducting gynecological or proctological exams, comprising:
a sensor body having a longitudinal axis, a proximal end, a distal end, a first passage disposed coaxial with the longitudinal axis and passing through the sensor body from the proximal end to the distal end, a second passage including a first opening disposed proximate the distal end of the sensor body, and a lip disposed on the distal end of the sensor body;
a grip disposed on the proximal end of the sensor body, the grip including a second opening of the second passage;
a brush control mechanism having a proximal end and a distal end, the proximal end of the brush control mechanism configured to pass through the first passage from the distal end of the sensor body; and
a brush disposed on the distal end of the brush control mechanism.
